(19) United States
(12) Patent Application Publication
(10) Pub. No.: US 2013/0224270 A1
(43) Pub. Date: Aug. 29, 2013

(54) Oral Care Composition Comprising Arginine and Calcium Carbonate

(71) Applicants: Richard Robinson, Belle Mead, NJ (US); Eric Simon, Somerset, NJ (US)

(72) Inventors: Richard Robinson, Belle Mead, NJ (US); Eric Simon, Somerset, NJ (US)

(73) Assignee: Colgate-Palmolive Company, New York, NY (US)

(21) Appl. No.: 13/881,516

(22) PCT Filed: Oct. 27, 2010
(86) PCT No.: PCT/US10/54185

§ 371 Date: Apr. 25, 2013

(51) Int. Cl. A61K 8/19; A61K 8/44; A61Q 11/00

(52) U.S. Cl. CPC A61K 8/19; A61Q 11/00; A61K 8/44
USPC 424/401; 424/54; 424/52

(57) ABSTRACT

This invention relates to oral care compositions comprising a basic amino acid or salt thereof, and an abrasive system comprising natural calcium carbonate and precipitated calcium carbonate; and to methods of using and of making these compositions.

### FIELD

This invention relates to oral care compositions comprising a basic amino acid or salt thereof, together with an improved calcium carbonate abrasive system, comprising natural calcium carbonate and precipitated calcium carbonate, and to methods of using and of making these compositions.

### BACKGROUND

Arginine and other basic amino acids have been proposed for use in oral care and are believed to have significant benefits in combating cavity formation and tooth sensitivity. Commercially available arginine-based toothpaste, such as ProClude® toothpaste or DenClude® toothpaste, for example, contains arginine bicarbonate and precipitated calcium carbonate, but not fluoride. The carbonate ion is believed to have cariostatic properties, and the calcium is believed to form in complex with arginine to provide a protective effect.

Natural calcium carbonate (e.g., chalk or limestone) typically has a well-defined crystal structure (making it very hard). It is generally quarried, must be milled to size. Natural calcium carbonate abrasives provide good tooth cleaning and stain removal, but they also highly abrasive, however, so that natural calcium carbonate product has been considered undesirable for persons having sensitive teeth. Precipitated calcium carbonate is more friable and less abrasive, resulting in less damaging abrasion to enamel, which is good for sensitive teeth, but also it typically provides less effective cleaning.

Accordingly, there is a need for a stable oral care product that comprising a basic amino acid and beneficial minerals such as fluoride and calcium, which moreover has an optimized abrasive system to provide effective cleaning without damaging abrasivity, particularly for people having sensitive teeth.

### BRIEF SUMMARY

It is now surprisingly discovered that a combination of natural calcium carbonate and high absorption precipitated calcium carbonate abrasives, together with a basic amino acid, e.g., arginine, provide a highly effective dentifrice, particularly for people having sensitive teeth. The invention thus encompasses oral care compositions and methods of using the same that are effective in inhibiting or reducing the accumulation of plaque, reducing levels of acid producing (cariogenic) bacteria, remineralizing teeth, inhibiting or reducing gingivitis, and reducing dentinal hypersensitivity. The invention also encompasses compositions and methods to clean the oral cavity and provide improved methods of promoting oral health and/or systemic health, including cardiovascular health, e.g., by reducing potential for systemic infection via the oral tissues.

The invention thus comprises an oral care composition (a Composition of the Invention), e.g., a dentifrice, comprising
(a) an effective amount of a basic amino acid in free or salt form, e.g., arginine bicarbonate;
(b) an abrasive comprising
(c) natural calcium carbonate (NCC) having an average particle size of 3-7 microns, e.g., about 5.5 microns and water absorption of 12-25 g/100 g, e.g. about 18 g/100 g; and
(d) precipitated calcium carbonate (PCC) having an average particle size of 1-5 microns, e.g. 2-3 microns, e.g. about 2.4 microns and water absorption of greater than 25 g/100 g;
(e) an effective amount of a fluoride source, e.g., a soluble fluoride salt, for example sodium monofluorophosphate.
   The composition is effective for cleaning and strengthening the teeth without damaging abrasion, e.g., in persons with sensitive teeth, for example has a good Pellicle Cleaning Ratio, e.g., at least 70, and a low Radioactive Dentine Abrasivity value, e.g., less than 140.

In some embodiments, the formulation further comprises an anionic surfactant, e.g., sodium lauryl sulfate; an anionic polymer, e.g., a copolymer of methyl vinyl ether and maleic anhydride; and/or an antibacterial agent, e.g., triclosan.

In particular embodiments, the Compositions of the Invention are in the form of a dentifrice comprising additional ingredients selected from one or more of water, abrasives, surfactants, foaming agents, vitamins, polymers, enzymes, humectants, thickeners, antimicrobial agents, preservatives, flavorings, colorings and/or combinations thereof.

Without intending to be bound by a particular theory, it is believed that the presence of small particles in a formulation with arginine and calcium may help plug the microtubules responsible for hypersensitive teeth and help repair precarious lesions in the enamel and dentin.

It is moreover found that the combination of fluoride and a basic amino acid, e.g., arginine, in an oral care product according to particular embodiments of the present invention produces unexpected benefits beyond and qualitatively different from what can be observed using compositions comprising effective amounts of either compound separately, in promoting remineralization, repairing pre-carious lesions, and enhancing oral health. It has moreover been found that this action can be further enhanced by addition of a small particle abrasive comprising a combination of natural calcium carbonate and precipitated calcium carbonate, which may act to help fill microfissures in the enamel and microtubules in the dentin.

The presence of a basic amino acid is also surprisingly found to reduce bacterial adhesion to the tooth surface, particularly when the basic amino acid is provided in combination with an anionic surfactant. The combination of the basic amino acid and the anionic surfactant and/or anionic polymer e.g., PVM/MA also enhances delivery of antimicrobial agents, particularly triclosan.

The invention thus further encompasses methods to (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing bacteria, (viii) to increase relative levels of arginolytic bacteria, (ix) inhibit microbial biofilm formation in the oral cavity, (x) raise and/or maintain plaque pH at levels of at least pH about 5.5 following sugar challenge, (xi) reduce plaque accumulation, (xii) reduce dry mouth, (xiii) reduce erosion, (xiv) whiten the teeth, (xv) immunize or protect the teeth against cariogenic bacteria, (xvi) clean the teeth and oral cavity and/or (xvii) promote systemic health, including cardiovascular health, e.g., by reducing potential for systemic infection via the oral tissues, comprising applying a Composition of the Invention to the oral cavity, e.g., by applying a Composition of the Invention to the oral cavity of a subject in need thereof.

### DETAILED DESCRIPTION

As used throughout, ranges are used as a shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls. In addition, the compositions and the methods may comprise, consist essentially of or consist of the elements described therein.

Unless otherwise specified, all percentages and amounts expressed herein and elsewhere in the specification should be understood to refer to percentages by weight. The amounts given are based on the active weight of the material. The recitation of a specific value herein is intended to denote that value, plus or minus a degree of variability to account for errors in measurements. For example, an amount of 10% may include 9.5% or 10.5%, given the degree of error in measurement that will be appreciated and understood by those having ordinary skill in the art.

The invention thus comprises a dentifrice composition (Composition 1.0) comprising
(a) an effective amount of a basic amino acid in free or salt form, e.g., arginine bicarbonate;
(b) an abrasive comprising
   (i) natural calcium carbonate (NCC) having an average particle size of 3-7 microns, e.g., about 5.5 microns and, e.g., water absorption of 12-25 g/100 g, e.g. about 18 g/100 g; and
   (ii) precipitated calcium carbonate (PCC) having an average particle size 1-5 microns, e.g. 2-3 microns, e.g. about 2.4 microns and, e.g. water absorption of greater than 25 g/100 g; and
(c) an effective amount of a fluoride source, e.g., a soluble fluoride salt, for example sodium monofluorophosphate.
   For example, the invention provides any of the following compositions:
1.0.1. Composition 1.0 having a Pellicle Cleaning Ratio, of at least 70, and a Radioactive Dentine Abrasivity value of less than 140.
1.0.2. Composition 1.0 or 1.0.1 wherein the basic amino acid is arginine, lysine, citrulline, ornithine, creatine, histidine, diaminobutanoic acid, diaminoproprionic acid, salts thereof and/or combinations thereof.
1.0.3. Any of the preceding compositions wherein the basic amino acid has the L-configuration.
1.0.4. Any of the preceding compositions is provided in the form of a salt of a di- or tri-peptide comprising the basic amino acid.
1.0.5. Any of the preceding compositions wherein the basic amino acid is arginine, e.g., L-arginine.
1.0.6. Any of the preceding compositions wherein the basic amino acid is partially or wholly in salt form.
1.0.7. Any of the preceding compositions wherein the basic amino acid comprises arginine phosphate.
1.0.8. Any of the preceding compositions wherein the basic amino acid comprises arginine hydrochloride.
1.0.9. Any of the preceding compositions wherein the basic amino acid comprises arginine sulfate.
1.0.10. Any of the preceding compositions wherein the basic amino acid comprises arginine bicarbonate.
1.0.11. Any of the preceding compositions wherein the salt of the basic amino acid is formed in situ in the formulation by neutralization of the basic amino acid with an acid or a salt of an acid.
1.0.12. Any of the preceding compositions wherein the salt of the basic amino acid is formed by neutralization of the basic amino acid to form a premix prior to combination with the fluoride salt.
1.0.13. Any of the preceding compositions wherein the basic amino acid is present in an amount corresponding to 1% to 15% of the total composition weight, the weight of the basic amino acid being calculated as free base form.
1.0.14. Any of the preceding compositions wherein the basic amino acid is present in an amount corresponding 8% to 10% of the total composition weight, the weight of the basic amino acid being calculated as free base form.
1.0.15. Any of the preceding compositions wherein the ratio of natural calcium carbonate to precipitated calcium carbonate is from 1:1 to 1:5, e.g., from 1:2 to 1:3, e.g, about 1:2.5.
1.0.16. Any of the preceding compositions wherein the natural calcium carbonate is a refined natural calcium carbonate.
1.0.17. Any of the preceding compositions wherein the natural calcium carbonate is a ground limestone product.
1.0.18. Any of the preceding composition wherein no more than 0.01%, preferably no more than 0.004% by weight of the natural calcium carbonate particles would not pass through a 325 mesh.
1.0.19. Any of the preceding compositions wherein the precipitated calcium carbonate has a D₅₀ of 2.3-2.7 microns, a D₉₀ of 3.7-5.0 microns and a D₁₀ of 1.1-1.5 microns.
1.0.20. Any of the preceding compositions wherein the natural calcium carbonate is present in an amount of 5%-20% by weight of the composition.
1.0.21. Any of the preceding compositions wherein the precipitated calcium carbonate is present in an amount of 10% to 35% by weight of the composition.
1.0.22. Any of the preceding compositions wherein the natural calcium carbonate is present in an amount of about 10% and the precipitated calcium carbonate is present in an amount of about 25% by weight of the composition.
1.0.23. Any of the preceding compositions wherein the fluoride source is a soluble fluoride salt.
1.0.24. Any of the preceding compositions wherein the fluoride source is a soluble fluoride salt selected from stannous fluoride, sodium fluoride, potassium fluoride, sodium monofluorophosphate, sodium fluorosilicate, ammonium fluorosilicate, amine fluoride (e.g., N′-octadecyltrimethylendiamine-N,N,N′-tris(2-ethanol)-dihydrofluoride), ammonium fluoride, titanium fluoride, hexafluorosulfate, and combinations thereof.
1.0.25. Any of the preceding compositions wherein the fluoride source is a fluorophosphate.
1.0.26. Any of the preceding composition wherein the fluoride source is sodium monofluorophosphate.
1.0.27. Any of the preceding compositions wherein the fluoride source is a fluoride salt present in an amount of 0.1 wt. % to 2 wt. % of the total composition weight.
1.0.28. Any of the preceding compositions wherein the fluoride source is a soluble fluoride salt present in the amount of about 1.1% by weight of the composition.
1.0.29. Any of the preceding compositions wherein the fluoride source is a soluble fluoride salt which provides fluoride ion in an amount of from about 50 to about 25,000 ppm.
1.0.30. Any of the preceding compositions wherein the fluoride source is a soluble fluoride salt which provides fluoride ion in an amount of about 750 to about 2000 ppm.
1.0.31. Any of the preceding compositions wherein the composition comprises about 1000 to about 1500 ppm fluoride ion.
1.0.32. Any of the preceding compositions wherein the composition comprises about 1450 ppm fluoride ion.
1.0.33. Any of the preceding compositions wherein the pH is between 6 and 9, e.g., 6.5-7.4 or 7.5-9.
1.0.34. Any of the preceding compositions wherein the pH is between 6.5 and 7.4.
1.0.35. Any of the preceding compositions wherein the pH is approximately neutral.
1.0.36. Any of the preceding compositions wherein the pH is 8.5-9.5.
1.0.37. Any of the preceding compositions further comprising an anti-calculus agent.
1.0.38. Any of the preceding compositions further comprising an anti-calculus agent which is a polyphosphate, e.g., pyrophosphate, tripolyphosphate, or hexametaphosphate, e.g., in sodium salt form.
1.0.39. Any of the preceding compositions comprising at least one surfactant.
1.0.40. Any of the preceding compositions comprising at least one surfactant selected from sodium lauryl sulfate, cocamidopropyl betaine, and combinations thereof.
1.0.41. Any of the preceding compositions comprising an anionic surfactant, e.g., selected from sodium lauryl sulfate, sodium laureth sulfate, and mixtures thereof.
1.0.42. Any of the preceding compositions comprising sodium lauryl sulfate, in an amount from 0.5-3% by wt of the composition.
1.0.43. Any of the preceding compositions comprising at least one humectant.
1.0.44. Any of the preceding compositions comprising at least one humectant selected from glycerin, sorbitol and combinations thereof.
1.0.45. Any of the preceding compositions comprising at least one polymer.
1.0.46. Any of the preceding compositions comprising at least one polymer selected from polyethylene glycols, polyvinylmethyl ether maleic acid copolymers, polysaccharides (e.g., cellulose derivatives, for example carboxymethyl cellulose, or polysaccharide gums, for example xanthan gum or carrageenan gum), and combinations thereof.
1.0.47. Any of the preceding compositions comprising gum strips or fragments.
1.0.48. Any of the preceding compositions comprising flavoring, fragrance and/or coloring.
1.0.49. Any of the preceding compositions comprising water.
1.0.50. Any of the preceding compositions comprising an antibacterial agent.
1.0.51. Any of the preceding compositions comprising an antibacterial agent selected from halogenated diphenyl ether (e.g. triclosan), herbal extracts and essential oils (e.g., rosemary extract, tea extract, magnolia extract, thymol, menthol, eucalyptol, geraniol, carvacrol, citral, hinokitol, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC)), phenolic antiseptics, hexetidine, octenidine, sanguinarine, povidone iodine, delmopinol, salifluor, metal ions (e.g., zinc salts, for example, zinc citrate, stannous salts, copper salts, iron salts), sanguinarine, propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol and other piperidino derivatives, nicin preparations, chlorite salts; and mixtures of any of the foregoing.
1.0.52. Any of the preceding compositions comprising an anti-inflammatory compound, e.g., an inhibitor of at least one of host pro-inflammatory factors selected from matrix metalloproteinases (MMP's), cyclooxygenases (COX), PGE₂, interleukin 1 (IL-1), IL-1β converting enzyme (ICE), transforming growth factor β1 (TGF-β1), inducible nitric oxide synthase (iNOS), hyaluronidase, cathepsins, nuclear factor kappa B (NF-κB), and IL-1 Receptor Associated Kinase (IRAK), e,g, selected from aspirin, ketorolac, flurbiprofen, ibuprofen, naproxen, indomethacin, aspirin, ketoprofen, piroxicam, meclofenamic acid, nordihydoguaiaretic acid, and mixtures thereof.
1.0.53. Any of the preceding compositions comprising an antioxidant, e.g., selected from the group consisting of Co-enzyme Q10, PQQ, Vitamin C, Vitamin E, Vitamin A, anethole-dithiothione, and mixtures thereof.
1.0.54. Any of the preceding compositions comprising triclosan.
1.0.55. Any of the preceding compositions comprising an antibacterial agent in an amount of about 0.01-about 5 wt. % of the total composition weight.
1.0.56. Any of the preceding compositions comprising triclosan in an amount of about 0.01 to about 1 wt. percent of the total composition weight.
1.0.57. Any of the preceding compositions comprising triclosan in an amount of about 0.3% of the total composition weight.
1.0.58. Any of the preceding compositions comprising triclosan and Zn²⁺ ion source e.g., zinc citrate.
1.0.59. Any of the preceding compositions comprising a whitening agent.
1.0.60. Any of the preceding compositions comprising a whitening agent selected from a whitening active selected from the group consisting of peroxides, metal chlorites, perborates, percarbonates, peroxyacids, hypochlorites, and combinations thereof
1.0.61. Any of the preceding compositions further comprising hydrogen peroxide or a hydrogen peroxide source, e.g., urea peroxide or a peroxide salt or complex (e.g., such as peroxyphosphate, peroxycarbonate, perborate, peroxysilicate, or persulphate salts; for example calcium peroxyphosphate, sodium perborate, sodium carbonate peroxide, sodium peroxyphosphate, and potassium persulfate), or hydrogen peroxide polymer complexes such as hydrogen peroxide-polyvinyl pyrrolidone polymer complexes.
1.0.62. Any of the preceding compositions further comprising a source of calcium and phosphate selected from (i) calcium-glass complexes, e.g., calcium sodium phosphosilicates, and (ii) calcium-protein complexes, e.g., casein phosphopeptide-amorphous calcium phosphate.
1.0.63. Any of the preceding compositions further comprising a soluble calcium salt, e.g., selected from calcium sulfate, calcium chloride, calcium nitrate, calcium acetate, calcium lactate, and combinations thereof.
1.0.64. Any of the preceding compositions further comprising an agent that interferes with or prevents bacterial attachment, e.g., solbrol or chitosan.
1.0.65. Any of the preceding compositions further comprising a physiologically acceptable potassium salt, e.g., potassium nitrate, potassium citrate, or potassium chloride, in an amount effective to reduce dentinal sensitivity.
1.0.66. Any of the preceding compositions comprising from about 0.1% to about 7.5% of a physiologically acceptable potassium salt, e.g., potassium nitrate and/or potassium chloride.
1.0.67. Any of the preceding compositions effective upon application to the oral cavity, e.g., with brushing, to (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing bacteria, (viii) to increase relative levels of arginolytic bacteria, (ix) inhibit microbial biofilm formation in the oral cavity, (x) raise and/or maintain plaque pH at levels of at least pH 5.5 following sugar challenge, (xi) reduce plaque accumulation, (xii) reduce dry mouth, (xiii) clean the teeth and oral cavity (xiv) reduce erosion, (xv) whiten teeth, and/or (xvi) immunize the teeth against cariogenic bacteria.
1.0.68. A composition obtained or obtainable by combining the ingredients as set forth in any of the preceding compositions.
1.0.69. Any of the preceding compositions wherein the composition is toothpaste.
1.0.70. Any of the preceding compositions wherein the composition is a toothpaste optionally further comprising one or more of one or more of water, abrasives, surfactants, foaming agents, vitamins, polymers, enzymes, humectants, thickeners, antimicrobial agents, preservatives, flavorings, colorings and/or combinations thereof.
1.0.71. Any of the preceding compositions comprising
   (a) an effective amount of a basic amino acid in free or salt form, e.g., arginine bicarbonate;
   (b) an abrasive comprising
      (i) natural calcium carbonate (NCC) having an average particle size of 3-7 microns, e.g., about 5.5 microns and water absorption of 12-25 g/100 g, e.g. about 18 g/100 g; and
      (ii) precipitated calcium carbonate (PCC) having an average particle size 1-5 microns, e.g. 2-3 microns, e.g. about 2.4 microns and water absorption of greater than 25 g/100 g;
   (c) an effective amount of a fluoride source, e.g., a soluble fluoride salt, for example sodium monofluorophosphate; and
   (d) an anionic surfactant, e.g., sodium lauryl sulfate.
1.0.72. A dentifrice according any of the preceding embodiments comprising
   (a) 3-15% w/w arginine bicarbonate;
   (b) an abrasive comprising
      (i) 5-15% w/w natural calcium carbonate (NCC) having an average particle size of 3-7 microns, e.g., about 5.5 microns and water absorption of 12-25 g/100 g, e.g. about 18 g/100 g; and
      (ii) 20-30% w/w precipitated calcium carbonate (PCC) having an average particle size 1-5 microns, e.g. 2-3 microns, e.g. about 2.4 microns and water absorption of greater than 25 g/100 g; and
      (iii) 0.1-2% w/w, e.g., about 1.1% w/w of sodium monofluorophosphate; and
      (iv) 0.5-3% w/w sodium lauryl sulfate.

The invention thus provides a toothpaste according to any of the foregoing compositions 1.0-1.0.72 comprising:

| Water | | |
|---|---|---|
| Humectants | 20-35 | wt % |
| Thickeners and polymers | 0-5 | wt % |
| Flavorings and pigments | 0.01-5 | wt % |
| Buffers | 0-3 | wt % |
| Soluble fluoride salt | 0.3-2 | wt % |
| Arginine bicarbonate | 5-12 | wt % |
| Natural calcium carbonate abrasive | 5-15 | wt % |
| Precipitated calcium carbonate abrasive | 15-35 | wt % |
| Surfactant | 0.3-3 | wt % |

In another embodiment, the invention encompasses a method ((Method 2) to improve oral health comprising applying an effective amount of the oral composition of any of the embodiments under Compositions 1.0-1.0.72. to the oral cavity of a subject in need thereof, e.g., a method to
(a) reduce or inhibit formation of dental caries,
(b) reduce, repair or inhibit pre-carious lesions of the enamel, e.g., as detected by quantitative light-induced fluorescence (QLF) or electrical caries measurement (ECM),
(c) reduce or inhibit demineralization and promote remineralization of the teeth,
(d) reduce hypersensitivity of the teeth,
(e) reduce or inhibit gingivitis,
(f) promote healing of sores or cuts in the mouth,
(g) reduce levels of acid producing bacteria,
(h) to increase relative levels of arginolytic bacteria,
(i) inhibit microbial biofilm formation in the oral cavity,
(j) raise and/or maintain plaque pH at levels of at least pH about 5.5 following sugar challenge,
(k) reduce plaque accumulation,
(l) reduce erosion,
(m) whiten teeth,
(n) enhance systemic health,
(o) immunize or protect teeth against cariogenic bacteria; and/or clean the teeth and oral cavity.

It may therefore be seen by the skilled practitioner in the oral care art that a surprising technical effect and advantage of enhanced dentinal occlusion of sensitive teeth can result from the formulation, and use, of an oral care composition, for example a dentifrice, in accordance with one or more aspects of the invention, which are directed to the provision of combinations of active components or ingredients, and preferably their respective amounts, within the composition.

Levels of active ingredients will vary based on the nature of the delivery system and the particular active. For example, the basic amino acid may be present at levels from, e.g., about 3 to about 112 wt % for a consumer toothpaste or about 7 to about 20 wt % for a professional or prescription treatment product (amount expressed as weight of free base). Fluoride may be present at levels of, e.g., about 750 to about 2,000 ppm for a consumer toothpaste, or about 2,000 to about 10,000 ppm for a professional or prescription treatment product. Levels of antibacterial will in accordance with the particular agent and formulation, e.g., a triclosan toothpaste may contain about 0.3 wt % triclosan.

### Measurements

RDA: RDA is an abbreviation for radioactive dentin abrasion, a relative measure of abrasivity. Typically, extracted human or cow teeth are irradiated in a neutron flux, mounted in methylmethacrylate (bone glue), stripped of enamel, inserted into a brushing-machine, brushed by American Dental Association (ADA) standards (reference toothbrush, 150 g pressure, 1500 strokes, 4-to-1 water-toothpaste slurry). The radioactivity of the rinse water is then measured and recorded. For experimental control, the test is repeated with an ADA reference toothpaste made of calcium pyrophosphate, with this measurement given a value of 100 to calibrate the relative scale. See, e.g., Hefferren, Journal of Dental Research, 55:4, 1976, 563-573, and U.S. Pat. Nos. 4,340,583; 4,420,312; and 4,421,527.

PCR or pellicle cleaning ratio is a measure of the effectiveness of the dentifrice to remove stains, e.g. described U.S. Pat. Nos. 5,658,553 and 5,651,958. Typically, a clear pellicle material is applied to a bovine tooth which is then stained with a combination of the pellicle material and tea, coffee, and FeCl₃, which is subsequently treated with the composition, and the change in the reflectance of the tooth surface before and after treatment is the PCR value.

### Basic Amino Acids

The basic amino acids which can be used in the compositions and methods of the invention include not only naturally occurring basic amino acids, such as arginine, lysine, and histidine, but also any basic amino acids having a carboxyl group and an amino group in the molecule, which are water-soluble and provide an aqueous solution with a pH of about 7 or greater.

Accordingly, basic amino acids include, but are not limited to, arginine, lysine, citrulline, ornithine, creatine, histidine, diaminobutanoic acid, diaminoproprionic acid, salts thereof or combinations thereof. In a particular embodiment, the basic amino acids are selected from arginine, citrulline, and ornithine. In certain embodiments, the basic amino acid is arginine, for example, 1-arginine, or a salt thereof.

The compositions of the invention are intended for topical use in the mouth and so salts for use in the present invention should be safe for such use, in the amounts and concentrations provided. Suitable salts include salts known in the art to be pharmaceutically acceptable salts are generally considered to be physiologically acceptable in the amounts and concentrations provided. Physiologically acceptable salts include those derived from pharmaceutically acceptable inorganic or organic acids or bases, for example acid addition salts formed by acids which form a physiological acceptable anion, e.g., hydrochloride or bromide salt, and base addition salts formed by bases which form a physiologically acceptable cation, for example those derived from alkali metals such as potassium and sodium or alkaline earth metals such as calcium and magnesium. Physiologically acceptable salts may be obtained using standard procedures known in the art, for example, by reacting a sufficiently basic compound such as an amine with a suitable acid affording a physiologically acceptable anion.

In various embodiments, the basic amino acid is present in an amount of about 0.5 wt. % to about 20 wt. % of the total composition weight, about 1 wt. % to about 10 wt. % of the total composition weight, for example about 1.5 wt. %, about 3.75 wt. %, about 5 wt. %, or about 7.5 wt. % of the total composition weight.

### Fluoride Ion Sources

The oral care compositions may further include one or more fluoride ion sources, e.g., soluble fluoride salts. A wide variety of fluoride ion-yielding materials can be employed as sources of soluble fluoride in the present compositions. Examples of suitable fluoride ion-yielding materials are found in U.S. Pat. No. 3,535,421, to Briner et al.; U.S. Pat. No. 4,885,155, to Parran, Jr. et al. and U.S. Pat. No. 3,678,154, to Widder et al., incorporated herein by reference.

Representative fluoride ion sources include, but are not limited to, stannous fluoride, sodium fluoride, potassium fluoride, sodium monofluorophosphate, sodium fluorosilicate, ammonium fluorosilicate, amine fluoride, ammonium fluoride, and combinations thereof. In certain embodiments the fluoride ion source includes stannous fluoride, sodium fluoride, sodium monofluorophosphate as well as mixtures thereof.

In certain embodiments, the oral care composition of the invention may also contain a source of fluoride ions or fluorine-providing ingredient in amounts sufficient to supply about 25 ppm to 25,000 ppm of fluoride ions, generally at least about 500 ppm, e.g., about 500 to about 2000 ppm, e.g., about 1000 to about 1600 ppm, e.g., about 1450 ppm. The appropriate level of fluoride will depend on the particular application. A toothpaste for general consumer use would typically have about 1000 to about 1500 ppm, with pediatric toothpaste having somewhat less. A dentifrice or coating for professional application could have as much as 5,000 or even 25,000 ppm fluoride.

Fluoride ion sources may be added to the compositions of the invention at a level of about 0.01 wt. % to about 10 wt. % in one embodiment or about 0.03 wt. % to about 5 wt. %, and in another embodiment about 0.1 wt. % to about 1 wt. % by weight of the composition in another embodiment. Weights of fluoride salts to provide the appropriate level of fluoride ion will obviously vary based on the weight of the counter ion in the salt.

Where the composition comprises calcium bicarbonate, sodium monofluorophosphate is preferred to sodium fluoride for stability reasons.

### Abrasives

Natural calcium carbonate is found in rocks such as chalk, limestone, marble and travertine. It is also the principle component of egg shells and the shells of mollusks. The natural calcium carbonate abrasive of the invention is typically a finely ground limestone which may optionally be refined or partially refined to remove impurities. For use in the present invention, the material has an average particle size of less than 10 microns, e.g., 3-7 microns, e.g. about 5.5 microns. Because natural calcium carbonate may contain a high proportion of relatively large particles of not carefully controlled, which may unacceptably increase the abrasivity, preferably no more than 0.01%, preferably no more than 0.004% by weight of particles would not pass through a 325 mesh. The material has strong crystal structure, and is thus much harder and more abrasive than precipitated calcium carbonate. The tap density for the natural calcium carbonate is for example between 1 and 1.5 g/cc, e.g., about 1.2 for example about 1.19 g/cc. There are different polymorphs of natural calcium carbonate, e.g., calcite, aragonite and vaterite, calcite being preferred for purposes of this invention. An example of a commercially available product suitable for use in the present invention includes Vicron® 25-11 FG from GMZ.

Precipitated calcium carbonate is generally made by calcining limestone, to make calcium oxide (lime), which can then be converted back to calcium carbonate by reaction with carbon dioxide in water. Precipitated calcium carbonate has a different crystal structure from natural calcium carbonate. It is generally more friable and more porous, thus having lower abrasivity and higher water absorption. For use in the present invention, the particles are small, e.g., having an average particle size of 1-5 microns, and e.g., no more than 0.1%, preferably no more than 0.05% by weight of particles which would not pass through a 325 mesh. The particles may for example have a D₉₀ of 3-6 microns, for example 3.8=4.9, e.g., about 4.3; a D₅₀ of 1-4 microns, e.g. 2.2-2.6 microns, e.g., about 2.4 microns, and a D₁₀ of 1-2 microns, e.g., 1.2-1.4, e.g. about 1.3 microns. The particles have relatively high water absorption, e.g., at least 25 g/100 g, e.g. 30-70 g/100 g. Examples of commercially available products suitable for use in the present invention include, for example, Carbolag® 15 Plus from Lagos Industria Quimica.

In certain embodiments the invention may comprise additional calcium-containing abrasives, for example calcium phosphate abrasive, e.g., tricalcium phosphate (Ca₃(PO₄)₂), hydroxyapatite (Ca₁₀(PO₄)₆(OH)₂), or dicalcium phosphate dihydrate (CaHPO₄.2H₂O, also sometimes referred to herein as DiCal) or calcium pyrophosphate, and/or silica abrasives such as precipitated silicas having a mean particle size of up to about 20 μm, such as Zeodent 115®, marketed by J. M. Huber, sodium metaphosphate, potassium metaphosphate, aluminum silicate, calcined alumina, bentonite or other siliceous materials, or combinations thereof. However the additional abrasives are preferably not present in a type or amount so as to increase the RDA of the dentifrice to levels which could damage sensitive teeth, e.g., greater than 130.

### Foaming Agents

The oral care compositions of the invention also may include an agent to increase the amount of foam that is produced when the oral cavity is brushed.

Illustrative examples of agents that increase the amount of foam include, but are not limited to polyoxyethylene and certain polymers including, but not limited to, alginate polymers.

The polyoxyethylene may increase the amount of foam and the thickness of the foam generated by the oral care carrier component of the present invention. Polyoxyethylene is also commonly known as polyethylene glycol (“PEG”) or polyethylene oxide. The polyoxyethylenes suitable for this invention will have a molecular weight of about 200,000 to about 7,000,000. In one embodiment the molecular weight will be about 600,000 to about 2,000,000 and in another embodiment about 800,000 to about 1,000,000. Polyox® is the trade name for the high molecular weight polyoxyethylene produced by Union Carbide.

The polyoxyethylene may be present in an amount of about 1% to about 90%, in one embodiment about 5% to about 50% and in another embodiment about 10% to about 20% by weight of the oral care carrier component of the oral care compositions of the present invention. The dosage of foaming agent in the oral care composition (i.e., a single dose) is about 0.01 to about 0.9% by weight, about 0.05 to about 0.5% by weight, and in another embodiment about 0.1 to about 0.2% by weight.

### Surfactants

Another agent optionally included in the oral care composition of the invention is a surfactant or a mixture of compatible surfactants. Suitable surfactants are those which are reasonably stable throughout a wide pH range, for example, anionic, cationic, nonionic or zwitterionic surfactants.

Suitable surfactants are described more fully, for example, in U.S. Pat. No. 3,959,458, to Agricola et al.; U.S. Pat. No. 3,937,807, to Haefele; and U.S. Pat. No. 4,051,234, to Gieske et al., which are incorporated herein by reference.

In certain embodiments, the anionic surfactants useful herein include the water-soluble salts of alkyl sulfates having about 10 to about 18 carbon atoms in the alkyl radical and the water-soluble salts of sulfonated monoglycerides of fatty acids having about 10 to about 18 carbon atoms. Sodium lauryl sulfate, sodium lauroyl sarcosinate and sodium coconut monoglyceride sulfonates are examples of anionic surfactants of this type. Mixtures of anionic surfactants may also be utilized.

In another embodiment, cationic surfactants useful in the present invention can be broadly defined as derivatives of aliphatic quaternary ammonium compounds having one long alkyl chain containing about 8 to about 18 carbon atoms such as lauryl trimethylammonium chloride, cetyl pyridinium chloride, cetyl trimethylammonium bromide, di-isobutylphenoxyethyldimethylbenzylammonium chloride, coconut alkyltrimethylammonium nitrite, cetyl pyridinium fluoride, and mixtures thereof.

Illustrative cationic surfactants are the quaternary ammonium fluorides described in U.S. Pat. No. 3,535,421, to Briner et al., herein incorporated by reference. Certain cationic surfactants can also act as germicides in the compositions.

Illustrative nonionic surfactants that can be used in the compositions of the invention can be broadly defined as compounds produced by the condensation of alkylene oxide groups (hydrophilic in nature) with an organic hydrophobic compound which may be aliphatic or alkylaromatic in nature. Examples of suitable nonionic surfactants include, but are not limited to, the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials.

In certain embodiments, zwitterionic synthetic surfactants useful in the present invention can be broadly described as derivatives of aliphatic quaternary ammonium, phosphomium, and sulfonium compounds, in which the aliphatic radicals can be straight chain or branched, and wherein one of the aliphatic substituents contains about 8 to about 18 carbon atoms and one contains an anionic water-solubilizing group, e.g., carboxy, sulfonate, sulfate, phosphate or phosphonate. Illustrative examples of the surfactants suited for inclusion into the composition include, but are not limited to, sodium alkyl sulfate, sodium lauroyl sarcosinate, cocoamidopropyl betaine and polysorbate 20, and combinations thereof.

In a particular embodiment, the Composition of the Invention comprises an anionic surfactant, e.g., sodium lauryl sulfate.

The surfactant or mixtures of compatible surfactants can be present in the compositions of the present invention in about 0.1% to about 5.0%, in another embodiment about 0.3% to about 3.0% and in another embodiment about 0.5% to about 2.0% by weight of the total composition.

### Flavoring Agents

The oral care compositions of the invention may also include a flavoring agent. Flavoring agents which are used in the practice of the present invention include, but are not limited to, essential oils as well as various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Certain embodiments employ the oils of peppermint and spearmint.

The flavoring agent is incorporated in the oral composition at a concentration of about 0.1 to about 5% by weight and about 0.5 to about 1.5% by weight. The dosage of flavoring agent in the individual oral care composition dosage (i.e., a single dose) is about 0.001 to about 0.05% by weight and in another embodiment about 0.005 to about 0.015% by weight.

### Chelating Agents

The oral care compositions of the invention also may optionally include one or more chelating agents able to complex calcium found in the cell walls of the bacteria. Binding of this calcium weakens the bacterial cell wall and augments bacterial lysis.

Another group of agents suitable for use as chelating agents in the present invention are the soluble pyrophosphates. The pyrophosphate salts used in the present compositions can be any of the alkali metal pyrophosphate salts. In certain embodiments, salts include tetra alkali metal pyrophosphate, dialkali metal diacid pyrophosphate, trialkali metal monoacid pyrophosphate and mixtures thereof, wherein the alkali metals are sodium or potassium. The salts are useful in both their hydrated and unhydrated forms. An effective amount of pyrophosphate salt useful in the present composition is generally enough to provide at least about 1.0 wt. % pyrophosphate ions, about 1.5 wt. % to about 6 wt. %, about 3.5 wt. % to about 6 wt. % of such ions.

### Polymers

The oral care compositions of the invention also optionally include one or more polymers, such as polyethylene glycols, polyvinylmethyl ether maleic acid copolymers, polysaccharides (e.g., cellulose derivatives, for example carboxymethyl cellulose, or polysaccharide gums, for example xanthan gum or carrageenan gum). Acidic polymers, for example polyacrylate gels, may be provided in the form of their free acids or partially or fully neutralized water soluble alkali metal (e.g., potassium and sodium) or ammonium salts. Certain embodiments include 1:4 to 4:1 copolymers of maleic anhydride or acid with another polymerizable ethylenically unsaturated monomer, for example, methyl vinyl ether (methoxyethylene) having a molecular weight (M.W.) of about 30,000 to about 1,000,000. These copolymers are available for example as Gantrez AN 139(M.W. 500,000), AN 119 W. 250,000) and S-97 Pharmaceutical Grade (M.W. 70,000), of GAF Chemicals Corporation.

Other operative polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

Suitable generally, are polymerized olefinically or ethylenically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility.

A further class of polymeric agents includes a composition containing homopolymers of substituted acrylamides and/or homopolymers of unsaturated sulfonic acids and salts thereof, in particular where polymers are based on unsaturated sulfonic acids selected from acrylamidoalykane sulfonic acids such as 2-acrylamide 2 methylpropane sulfonic acid having a molecular weight of about 1,000 to about 2,000,000, described in U.S. Pat. No. 4,842,847, Jun. 27, 1989 to Zahid, incorporated herein by reference.

Another useful class of polymeric agents includes polyamino acids, particularly those containing proportions of anionic surface-active amino acids such as aspartic acid, glutamic acid and phosphoserine, as disclosed in U.S. Pat. No. 4,866,161 Sikes et al., incorporated herein by reference.

In preparing oral care compositions, it is sometimes necessary to add some thickening material to provide a desirable consistency or to stabilize or enhance the performance of the formulation. In certain embodiments, the thickening agents are carboxyvinyl polymers, carrageenan, hydroxyethyl cellulose and water soluble salts of cellulose ethers such as sodium carboxymethyl cellulose and sodium carboxymethyl hydroxyethyl cellulose. Natural gums such as karaya, gum arabic, and gum tragacanth can also be incorporated. Colloidal magnesium aluminum silicate or finely divided silica can be used as component of the thickening composition to further improve the composition's texture. In certain embodiments, thickening agents in an amount of about 0.5% to about 5.0% by weight of the total composition are used.

### Enzymes

The oral care compositions of the invention may also optionally include one or more enzymes. Useful enzymes include any of the available proteases, glucanohydrolases, endoglycosidases, amylases, mutanases, lipases and mucinases or compatible mixtures thereof. In certain embodiments, the enzyme is a protease, dextranase, endoglycosidase and mutanase. In another embodiment, the enzyme is papain, endoglycosidase or a mixture of dextranase and mutanase. Additional enzymes suitable for use in the present invention are disclosed in U.S. Pat. No. 5,000,939 to Dring et al., U.S. Pat. No. 4,992,420; U.S. Pat. No. 4,355,022; U.S. Pat. No. 4,154,815; U.S. Pat. No. 4,058,595; U.S. Pat. No. 3,991,177; and U.S. Pat. No. 3,696,191 all incorporated herein by reference. An enzyme of a mixture of several compatible enzymes in the current invention constitutes about 0.002% to about 2.0% in one embodiment or about 0.05% to about 1.5% in another embodiment or in yet another embodiment about 0.1% to about 0.5%.

### Water

Water may also be present in the oral compositions of the invention. Water, employed in the preparation of commercial oral compositions should be deionized and free of organic impurities. Water commonly makes up the balance of the compositions and includes about 10% to about 90%, about 20% to about 60% or about 10% to about 30% by weight of the oral compositions. This amount of water includes the free water which is added plus that amount which is introduced with other materials such as with sorbitol or any components of the invention.

### Humectants

Within certain embodiments of the oral compositions, it is also desirable to incorporate a humectant to prevent the composition from hardening upon exposure to air. Certain humectants can also impart desirable sweetness or flavor to dentifrice compositions. The humectant, on a pure humectant basis, generally includes about 15% to about 70% in one embodiment or about 30% to about 65% in another embodiment by weight of the dentifrice composition.

Suitable humectants include edible polyhydric alcohols such as glycerine, sorbitol, xylitol, propylene glycol as well as other polyols and mixtures of these humectants. Mixtures of glycerin and sorbitol may be used in certain embodiments as the humectant component of the toothpaste compositions herein.

In addition to the above described components, the embodiments of this invention can contain a variety of optional dentifrice ingredients some of which are described below. Optional ingredients include, for example, but are not limited to, adhesives, sudsing agents, flavoring agents, sweetening agents, additional antiplaque agents, abrasives, and coloring agents. These and other optional components are further described in U.S. Pat. No. 5,004,597, to Majeti; U.S. Pat. No. 3,959,458 to Agricola et al. and U.S. Pat. No. 3,937,807, to Haefele, all being incorporated herein by reference.

### Methods of Manufacture

The compositions of the present invention can be made using methods which are common in the oral product area.

In one illustrative embodiment, the oral care composition is made by neutralizing or partially neutralizing arginine in a gel phase with an acid, e.g., phosphoric acid, hydrochloric acid or carbonic acid, and mixing. Humectant plus such as, for example, vitamins, fluoride are mixed separately. Then the abrasives and the arginine are added to the humectant mix. Then surfactants and flavorings are mixed in, and the final slurry is formed into a dentifrice product.

### Composition Use

The present invention in its method aspect involves applying to the oral cavity a safe and effective amount of the compositions described herein.

The compositions and methods according to the invention are useful to a method to protect the teeth by facilitating repair and remineralization, in particular to reduce or inhibit formation of dental caries, reduce or inhibit demineralization and promote remineralization of the teeth, reduce hypersensitivity of the teeth, and reduce, repair or inhibit pre-carious lesions of the enamel, e.g., as detected by quantitative light-induced fluorescence (QLF) or electronic caries monitor (ECM).

Quantitative Light-induced Fluorescence is a visible light fluorescence that can detect early lesions and longitudinally monitor the progression or regression. Normal teeth fluoresce in visible light; demineralized teeth do not or do so only to a lesser degree. The area of demineralization can be quantified and its progress monitored. Blue laser light is used to make the teeth auto fluoresce. Areas that have lost mineral have lower fluorescence and appear darker in comparison to a sound tooth surface. Software is used to quantify the fluorescence from a white spot or the area/volume associated with the lesion. Generally, subjects with existing white spot lesions are recruited as panelists. The measurements are performed in vivo with real teeth. The lesion area/volume is measured at the beginning of the clinical. The reduction (improvement) in lesion area/volume is measured at the end of 6 months of product use. The data is often reported as a percent improvement versus baseline.

Electrical Caries Monitoring is a technique used to measure mineral content of the tooth based on electrical resistance. Electrical conductance measurement exploits the fact that the fluid-filled tubules exposed upon demineralization and erosion of the enamel conduct electricity. As a tooth loses mineral, it becomes less resistive to electrical current due to increased porosity. An increase in the conductance of the patient's teeth therefore may indicate demineralization. Generally, studies are conducted of root surfaces with an existing lesion. The measurements are performed in vivo with real teeth. Changes in electrical resistance before and after 6 month treatments are made. In addition, a classical caries score for root surfaces is made using a tactile probe. The hardness is classified on a three point scale: hard, leathery, or soft. In this type of study, typically the results are reported as electrical resistance (higher number is better) for the ECM measurements and an improvement in hardness of the lesion based on the tactile probe score.

The Compositions of the Invention are thus useful in a method to reduce pre-carious lesions of the enamel (as measured by QLF or ECM) relative to a composition lacking effective amounts of fluorine and/or arginine.

The Compositions of the invention are additionally useful in methods to reduce harmful bacteria in the oral cavity, for example methods to reduce or inhibit gingivitis, reduce levels of acid producing bacteria, to increase relative levels of arginolytic bacteria, inhibit microbial biofilm formation in the oral cavity, raise and/or maintain plaque pH at levels of at least pH 5.5 following sugar challenge, reduce plaque accumulation, and/or clean the teeth and oral cavity.

Finally, by increasing the pH in the mouth and discouraging pathogenic bacteria, the Compositions of the Invention are useful to promote healing of sores or cuts in the mouth.

Enhancing oral health also provides benefits in systemic health, as the oral tissues can be gateways for systemic infections. Good oral health is associated with systemic health, including cardiovascular health. The compositions and methods of the invention provide particular benefits because basic amino acids, especially arginine, are sources of nitrogen which supply NO synthesis pathways and thus enhance microcirculation in the oral tissues. Providing a less acidic oral environment is also helpful in reducing gastric distress and creates an environment less favorable to Heliobacter, which is associated with gastric ulcers. Arginine in particular is required for high expression of specific immune cell receptors, for example T-cell receptors, so that arginine can enhance an effective immune response. The compositions and methods of the invention are thus useful to enhance systemic health, including cardiovascular health.

The compositions and methods according to the invention can be incorporated into oral compositions for the care of the mouth and teeth such as toothpastes, transparent pastes, gels, mouth rinses, sprays and chewing gum.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls. It is understood that when formulations are described, they may be described in terms of their ingredients, as is common in the art, notwithstanding that these ingredients may react with one another in the actual formulation as it is made, stored and used, and such products are intended to be covered by the formulations described.

The following examples further describe and demonstrate illustrative embodiments within the scope of the present invention. The examples are given solely for illustration and are not to be construed as limitations of this invention as many variations are possible without departing from the spirit and scope thereof. Various modifications of the invention in addition to those shown and described herein should be apparent to those skilled in the art and are intended to fall within the appended claims.

### EXAMPLES

### Example 1

### Comparison of Different Abrasive Systems

Different combinations of abrasives are tested:
Formulation A: Prophylactic paste:

31% Sylodent 756,

15% Vicron 25-11 (fine ground CaCO3, natural source),

14% Vicron 41-8 (fine ground CaCO₃),

10% Arginine Bicarbonate
RDA: 230
Formulation B: Consumer sensitivity dentifrice

50% Vicron 25-11 (fine ground CaCO₃),

7%, Sylodent 15,

2% Arginine Bicarbonate
RDA: 179
   Additional formulations are prepared as depicted on Table 1, and the RDA and PCR measured:

TABLE 1

| | | RDA | PCR |
|---|---|---|---|
| C. | 8% Arginine/15% PCC/25% HA PCC | 66 | 62 |
| D. | 8% Arginine/35% Wolkem RNCC | 178 | 88 |
| E. | 8% Arginine/35% Vicron 25-11 NCC | 176 | 109 |
| F. | 8% Arginine/7% PCC/25% HA PCC/3% AC43 HCS | 172 | 94 |
| G. | 8% Arginine/10% Vicron 25-11 NCC/25% HA PCC | 102 | 87 |
| H. | 8% Arginine/10% Wolkem RNCC/25% HA PCC | 98 | 81 |

Initial formulation efforts adding 3% AC 43 high cleaning silica or replacing HA PCC (high absorption precipitated calcium carbonate) with NCC (natural calcium carbonate) abrasive result in RDAs of 170 and greater, compared to RDA of 110-130 for commercially available toothpaste for sensitive teeth. Other grades of NCC are tested, including an RNCC (refined natural calcium carbonate) from Wolkem, Inc of India, which did not provide sufficient cleaning efficacy at lower concentrations. The best combination is the Vicron 25-11 FG NCC at 10% by weight, having an average particle size of about 5.5 microns, in combination with high absorption Lagos PCC at 25% by weight, having an average particle size (D₅₀) of about 2.4, which resulted in a formulation that had both greater cleaning efficacy and low RDA.

### Example 2

### Optimized Dentifrice Formulation

Based on the preliminary testing with different abrasive combinations, an optimized dentifrice is prepared (Table 2) providing good cleaning and stain removal but low abrasivity:

| Ingredient | Wt % |
|---|---|
| Water | 25.225 |
| 70% Sorbitol | 23.000 |
| Carboxymethylcellulose | 0.720 |
| Xanthan | 0.135 |
| Acesulfame K | 0.400 |
| Sucralose | 0.020 |
| Sodium bicarbonate | 0.500 |
| Sodium silicate | 0.800 |
| Titanium dioxide | 0.500 |
| Sodium monofluorophosphate | 1.100 |
| Arginine bicarbonate | 10.000 |
| NCC - Vicron 25-11 | 10.000 |
| PCC - High Absorption | 25.000 |
| Sodium Lauryl Sulfate (SLS) | 1.500 |
| Flavor | 1.100 |
| TOTAL | 100.000 |

1. A dentifrice composition comprising
a. an effective amount of a basic amino acid in free or salt form;
b. an abrasive comprising
i. natural calcium carbonate (NCC) having an average particle size of 3-7 microns; and
ii. precipitated calcium carbonate (PCC);
wherein the composition has a Pellicle Cleaning Ratio (PCR), of at least 70, and a Radioactive Dentine Abrasivity (RDA) value of less than 140 and the ratio of natural calcium carbonate to precipitated calcium carbonate is from 1:1 to 1:5.

2. The composition according to claim 1 having precipitated calcium carbonate (PCC) having an average particle size of 1-5 microns.

3. (canceled)

4. The composition according to claim 1, further comprising an effective amount of a fluoride source.

5. The composition according to claim 1, wherein the basic amino acid in free or salt form is arginine bicarbonate.

6. The composition according to claim 1, wherein the basic amino acid is present in an amount corresponding to 1% to 15% of the total composition weight, the weight of the basic amino acid being calculated as free base form.

7. (canceled)

8. The composition according to claim 1 wherein the ratio of natural calcium carbonate to precipitated calcium carbonate is from 1:2 to 1:3.

9. The composition according to claim 1, wherein the natural calcium carbonate is present in an amount of 5%-20% by weight of the composition and the precipitated calcium carbonate is present in an amount of 10% to 35% by weight of the composition.

10. (canceled)

11. The composition according to claim 9, wherein the natural calcium carbonate is present in an amount of about 10% and the precipitated calcium carbonate is present in an amount of about 25% by weight of the composition.

12. The composition according to claim 1, further comprising an anionic surfactant.

13. The composition according to claim 1, further comprising an antibacterial agent.

14. The composition according to claim 1, further comprising an anionic polymer.

15. The composition according to claim 1, in the form of a toothpaste further comprising one or more of water, abrasives, surfactants, foaming agents, vitamins, polymers, enzymes, humectants, thickeners, antimicrobial agents, preservatives, flavorings, colorings and/or combinations thereof.

16. The composition according to claim 1, wherein the precipitated calcium carbonate (PCC) has a water absorption of greater than 25 g/100 g.

17. The composition according to claim 1, comprising the following ingredients:

| Water | | |
|---|---|---|
| Humectants | 20-35 | wt % |
| Thickeners and polymers | 0-5 | wt % |
| Flavorings and pigments | 0.01-5 | wt % |
| Buffers | 0-3 | wt % |
| Soluble fluoride salt | 0.3-2 | wt % |
| Arginine bicarbonate | 5-12 | wt % |
| Natural calcium carbonate abrasive | 5-15 | wt % |
| Precipitated calcium carbonate abrasive | 15-35 | wt % |
| Surfactant | 0.3-3 | wt % |

18. A method comprising applying an effective amount of the oral care composition of claim 1 to the oral cavity of a subject in need thereof, to
a. reduce or inhibit formation of dental caries,
b. reduce, repair or inhibit pre-carious lesions of the enamel,
c. reduce or inhibit demineralization and promote remineralization of the teeth,
d. reduce hypersensitivity of the teeth,
e. reduce or inhibit gingivitis,
f. promote healing of sores or cuts in the mouth,
g. reduce levels of acid producing bacteria,
h. to increase relative levels of arginolytic bacteria,
i. inhibit microbial biofilm formation in the oral cavity,
j. raise and/or maintain plaque pH at levels of at least pH 5.5 following sugar challenge,
k. reduce plaque accumulation,
l. treat, reduce, relieve or alleviate dry mouth,
m. whiten teeth,
n. reduce erosion,
o. promote systemic health,
p. immunize teeth against cariogenic bacteria; and/or
q. clean teeth and oral cavity.

19. (canceled)

20. The composition according to claim 1, wherein the basic amino acid in free or salt form is arginine bicarbonate and is present in an amount corresponding to 1% to 15% of the total composition weight, the weight of the arginine bicarbonate being calculated as free base form and the ratio of natural calcium carbonate to precipitated calcium carbonate is from 1:2 to 1:3.

21. The composition according to claim 1, wherein the basic amino acid in free or salt form is arginine bicarbonate, the natural calcium carbonate is present in an amount of 5%-20% by weight of the composition, the precipitated calcium carbonate is present in an amount of 10% to 35% by weight of the composition and the ratio of natural calcium carbonate to precipitated calcium carbonate is from 1:2 to 1:3.

22. The composition according to claim 20, further comprising an effective amount of a fluoride source.

23. The composition according to claim 21, further comprising an effective amount of a fluoride source.
